(19) United States
(12) Patent Application Publication
(10) Pub. No.: US 2024/0257956 A1
(43) Pub. Date: Aug. 1, 2024

(54) METHODS AND SYSTEMS FOR PREDICTING DELAYS IN EMERGENCY DEPARTMENT WORKFLOW

(71) Applicant: KONINKLIJKE PHILIPS N.V., EINDHOVEN (NL)

(72) Inventor: Ricardo da Silva Santos, Barueri (BR)

(21) Appl. No.: 18/425,509

(22) Filed: Jan. 29, 2024

Related U.S. Application Data
(60) Provisional application No. 63/442,584, filed on Feb. 1, 2023.

(51) Int. Cl. G16H 40/20; G06Q 10/0633; G06Q 10/0637; G16H 10/60

(52) U.S. Cl. CPC G16H 40/20; G06Q 10/0633; G06Q 10/06375; G16H 10/60

(57) ABSTRACT

A method for providing a delay prediction for emergency department (ED) workflow, comprising: (i) receiving patient information about one or more patients in the ED; (ii) receiving patient flow information comprising data about patient flow in the ED including information about execution of one or more past, current, and/or future workflow tasks for the one or more patients in the ED and one or more parameters for both resources utilized in workflow task execution and target time for workflow task execution; (iii) receiving resource information comprising data about resources for the ED; (iv) analyzing, with a trained workflow delay prediction algorithm, the received information to generate a predicted delay for ED workflow; and (v) providing, via a graphical user interface, the generated predicted delay.

### FIELD OF THE DISCLOSURE

The present disclosure is directed generally to methods and systems for providing delay predictions for emergency department (ED) workflow.

### BACKGROUND

The large number of patients in emergency departments (ED) is a worldwide problem. This results in long queues and wait times, treatment delays, increased patient length of stay (LOS) and, consequently, poor care quality.

To mitigate the problems caused by large numbers of patients, LOS time targets have been established. Some countries established or adopted a target of four hours from admission to discharge or similar action, while other countries established or adopted a target of six hours. One of the main strategies utilized to accomplish these targets is the early identification of bottlenecks in the ED operational workflow.

For example, process mining has been utilized to analyze the operational workflow of EDs in order to identify bottlenecks in steps of the ED workflow. Process mining is a computational technology that aims to discover, monitor, and improve real processes based on knowledge extracted from event records available in information systems. Although previous research has developed computational models for the identification of bottlenecks in the ED, there is a lack of research investigating the root cause of these bottlenecks.

From an observational study done in a medium-sized private general hospital, it is hereby discovered that one of the root causes of bottlenecks in the operational flow are individual delays occurring in one or more ED workflow tasks. These delays were due to interference from external or internal phenomena influencing the workflow task. Much of the interference from external phenomena is difficult to predict. For example, there can be sudden and unpredictable demand resulting from epidemic outbreaks or natural catastrophe. However, there are interferences from external phenomena that can be predicted. For example, observational study revealed that ED demand is much higher on Mondays. In contrast, interference by internal phenomena are generally more identifiable and measurable, thus allowing their prediction. For example, the absence of one or more physicians will likely imply a delay in clinical evaluations. However, existing tools for predicting delays in ED workflow are insufficient to prevent or alleviate those delays.

### SUMMARY OF THE DISCLOSURE

Accordingly, there is a continued need for methods and systems that predict, in real-time, potential delays in emergency department (ED) workflow tasks that can contribute to bottlenecks in the ED general workflow. Various embodiments and implementations herein are directed to an emergency department workflow analysis system configured to generate delay predictions for ED workflow. The system receives information from one or more databases, including patient information, patient flow information, parameters for resources utilized in workflow task execution and target time for workflow task execution, and resource information comprising data about resources for the ED. A trained workflow delay prediction algorithm analyzes the received information in order to generate a predicted delay in one or more current or future workflow tasks for the one or more patients, where the generated predicted delay is a department-level delay or a patient-level delay. The system provides, via a graphical user interface, a generated predicted delay, and stores that generated predicted delay for subsequent analysis.

Generally, in one aspect, a method for providing a delay prediction for emergency department (ED) workflow is provided. The method includes: (i) receiving, from an electronic medical record database, patient information about one or more patients in the ED; (ii) receiving, from an ED workflow database, (a) patient flow information comprising data about patient flow in the ED including information about execution of one or more past, current, and/or future workflow tasks for the one or more patients in the ED and (b) one or more parameters for both resources utilized in workflow task execution and target time for workflow task execution; (iii) receiving, from a resources database, resource information comprising data about resources for the ED; (iv) analyzing, with a trained workflow delay prediction algorithm, the received patient information, received patient flow information, received one or more parameters, and received resource information, to generate a predicted delay in one or more of the current or future workflow tasks for the one or more patients, wherein the generated predicted delay is a department-level delay or a patient-level delay; and (v) providing, via a graphical user interface, the generated predicted delay.

According to an embodiment, the method further comprises: generating, by the trained workflow delay prediction algorithm, one or more recommendations for preventing or alleviating the generated predicted delay; providing, via the graphical user interface, the one or more recommendations; and administering the one or more recommendations to prevent or alleviate the generated predicted delay.

According to an embodiment, the method further comprises storing, in a database, the generated predicted delay for subsequent analysis.

According to an embodiment, the one or more parameters for the resources utilized in workflow task execution are predetermined by a user. According to an embodiment, the one or more parameters for the target time for workflow task execution are predetermined by a user.

According to an embodiment, the provided generated predicted delay further comprises a graphic representation of workflow within the ED, and wherein the generated predicted delay is overlayed on the graphic representation at a location of the predicted delay within the workflow. According to an embodiment, the overlay of the generated predicted delay on the graphic representation comprises a color indicating the generated predicted delay.

According to an embodiment, the provided generated predicted delay further comprises a graphic representation of each of a plurality of patients in the ED, and further comprises an indication to which of the plurality of patients in the ED the generated predicted delay applies.

According to another aspect is a method for administering one or more recommendations to prevent or alleviate a delay prediction for emergency department (ED) workflow. The method includes: (i) receiving, via a graphical user interface, a generated predicted delay in ED workflow and one or more recommendations for preventing or alleviating the generated predicted delay in ED workflow, wherein the predicted delay in ED workflow and one or more recommendations are generated by: receiving, from an electronic medical record database, patient information about one or more patients in the ED; receiving, from an ED workflow database, (a) patient flow information comprising data about patient flow in the ED including information about execution of one or more past, current, and/or future workflow tasks for the one or more patients in the ED and (b) one or more parameters for both resources utilized in workflow task execution and target time for workflow task execution; receiving, from a resources database, resource information comprising data about resources for the ED; analyzing, with a trained workflow delay prediction algorithm, the received patient information, received patient flow information, received one or more parameters, and received resource information, to generate (a) a predicted delay in ED workflow and (b) one or more recommendations for preventing or alleviating the generated predicted delay in ED workflow; and (ii) administering the one or more recommendations to prevent or alleviate the generated predicted delay in ED workflow.

According to another aspect is a system for providing a delay prediction for emergency department (ED) workflow. The system includes: an electronic medical record database comprising patient information about one or more patients in the ED; an ED workflow database comprising: (i) patient flow information comprising data about patient flow in the ED including information about execution of one or more past, current, and/or future workflow tasks for the one or more patients in the ED and (ii) one or more parameters for both resources utilized in workflow task execution and target time for workflow task execution; a resources database comprising resource information comprising data about resources for the ED; a trained workflow delay prediction algorithm; a processor configured to: (i) receive patient information, patient flow information, one or more parameters, and resource information; (ii) analyze, using the trained workflow delay prediction algorithm, the received patient information, received patient flow information, received one or more parameters, and received resource information, to generate a predicted delay in one or more of the current or future workflow tasks for the one or more patients, wherein the generated predicted delay is a department-level delay or a patient-level delay; and a user interface configured to provide the generated predicted delay.

According to an embodiment, the processor is further configured to: (i) generate, using the trained workflow delay prediction algorithm, one or more recommendations for preventing or alleviating the generated predicted delay; and (ii) administer the one or more recommendations to prevent or alleviate the generated predicted delay.

According to an embodiment, the processor is further configured to store, in a database, the generated predicted delay for subsequent analysis.

According to an embodiment, the user interface is further configured to provide a graphic representation of workflow within the ED, wherein the generated predicted delay is overlayed on the graphic representation at a location of the predicted delay within the workflow. According to an embodiment, the overlay of the generated predicted delay on the graphic representation comprises a color indicating the generated predicted delay.

According to an embodiment, the user interface is further configured to provide a graphic representation of each of a plurality of patients in the ED, and an indication to which of the plurality of patients in the ED the generated predicted delay applies.

It should be appreciated that all combinations of the foregoing concepts and additional concepts discussed in greater detail below (provided such concepts are not mutually inconsistent) are contemplated as being part of the inventive subject matter disclosed herein. In particular, all combinations of claimed subject matter appearing at the end of this disclosure are contemplated as being part of the inventive subject matter disclosed herein. It should also be appreciated that terminology explicitly employed herein that also may appear in any disclosure incorporated by reference should be accorded a meaning most consistent with the particular concepts disclosed herein.

These and other aspects of the various embodiments will be apparent from and elucidated with reference to the embodiment(s) described hereinafter.

### BRIEF DESCRIPTION OF THE DRAWINGS

In the drawings, like reference characters generally refer to the same parts throughout the different views. The figures showing features and ways of implementing various embodiments and are not to be construed as being limiting to other possible embodiments falling within the scope of the attached claims. Also, the drawings are not necessarily to scale, emphasis instead generally being placed upon illustrating the principles of the various embodiments.

FIG. 1 is a flowchart of a method for providing a delay prediction for emergency department (ED) workflow, in accordance with an embodiment.

FIG. 2 is a schematic representation of an emergency department workflow analysis system, in accordance with an embodiment.

FIG. 3 is a flowchart of ED workflow, in accordance with an embodiment.

FIG. 4 is a schematic representation of an ED workflow analysis system, in accordance with an embodiment.

FIG. 5 is a schematic representation of patient data, in accordance with an embodiment.

FIG. 6 is a schematic representation of an ED workflow database and representative data, in accordance with an embodiment.

FIG. 7A is an interface for setting one or more parameters of the system, in accordance with an embodiment.

FIG. 7B is an interface for setting one or more parameters of the system, in accordance with an embodiment.

FIG. 8 is a flowchart of a method for training a workflow delay prediction algorithm, in accordance with an embodiment.

FIG. 9 is an interface for a data synchronization component, in accordance with an embodiment.

FIG. 10 is a schematic flowchart of the functionality of a trained workflow delay prediction algorithm, in accordance with an embodiment.

FIG. 11 is a schematic flowchart of prediction of delays in ED workflow caused by lab and imaging tests, in accordance with an embodiment.

FIG. 12 is a potential scheme for a rule-based algorithm, in accordance with an embodiment.

FIG. 13 is a possible embodiment of a graphical display of a generated predicted ED workflow delay, in accordance with an embodiment.

FIG. 14 is a patient view of a graphical display, in accordance with one embodiment.

### DETAILED DESCRIPTION OF EMBODIMENTS

The present disclosure describes various embodiments of a system and method configured to generate and provide delay predictions for emergency department (ED) workflow. More generally, Applicant has recognized and appreciated that it would be beneficial to provide information to clinicians about ED workflow in order to improve patient outcomes and maximize facility resources. Accordingly, an emergency department workflow analysis system is configured to receive information from one or more databases, including patient information, patient flow information, parameters for resources utilized in workflow task execution and target time for workflow task execution, and resource information comprising data about resources for the ED. A trained workflow delay prediction algorithm analyzes the received information in order to generate a predicted delay in one or more current or future workflow tasks for the one or more patients, where the generated predicted delay is a department-level delay or a patient-level delay. The system provides, via a graphical user interface, a generated predicted delay, and stores that generated predicted delay for subsequent analysis.

According to an embodiment, the methods and systems described or otherwise envisioned herein enable clinicians to better predict or determine workflow delays in an emergency department, at either a department level or a patient level. Thus, the methods and systems described or otherwise envisioned herein enable the emergency department to manage resources and implement recommendations or other changes to prevent or alleviate workflow delays. The trained workflow delay prediction algorithm can be implemented at real-time and can update frequently in order to provide up-to-the-minute information about workflow within an emergency department.

According to an embodiment, the methods and systems described or otherwise envisioned herein provides numerous advantages over the prior art. updated frequently or to be triggered at key decision points during patient's stay. Providing insight into predicted ED delays and roadblocks, and/or providing insight into the causes of those delays and roadblocks, enables recommendations and implementations to prevent predicted ED delays or even alleviate current ED delays. This directly results in improved patient outcomes and more efficient utilization of facility resources. Both improved patient outcomes and more efficient utilization of facility resources leads to saved lives.

The embodiments and implementations disclosed or otherwise envisioned herein can be utilized with any patient care system, including but not limited to clinical decision support tools, among other systems. For example, one application of the embodiments and implementations herein is to improve analysis systems such as, e.g., the Philips® IntelliSpace® and Patient Flow Capacity Suite (PFCS) products (manufactured by Koninklijke Philips, N.V.), among many other products. However, the disclosure is not limited to these devices or systems, and thus disclosure and embodiments disclosed herein can encompass any device or system capable of generating and providing delay predictions for ED workflow.

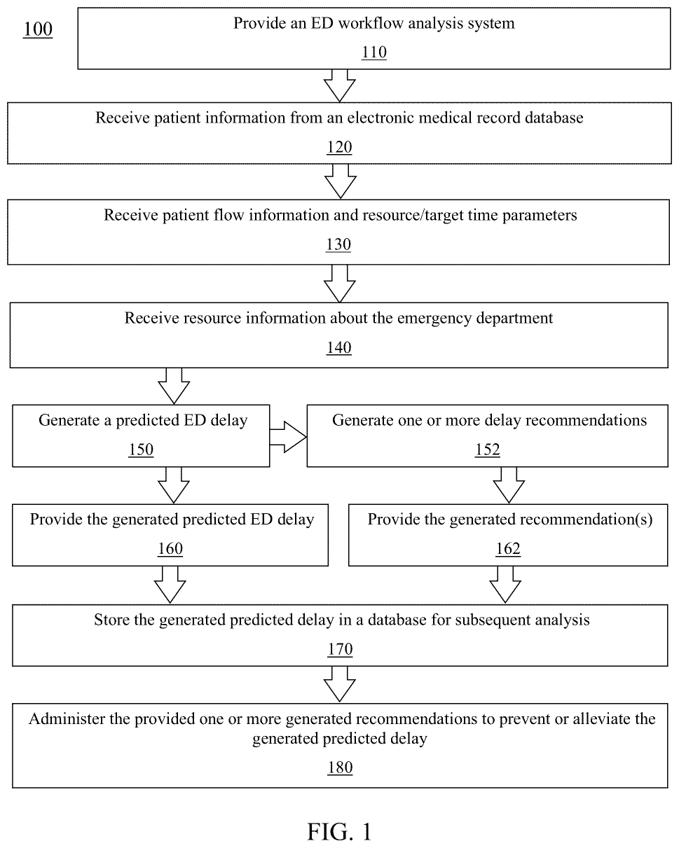

Referring to FIG. 1, in one embodiment, is a flowchart of a method 100 for generating and providing a delay prediction for ED workflow using an emergency department workflow analysis system. The methods described in connection with the figures are provided as examples only, and shall be understood not to limit the scope of the disclosure. The emergency department workflow analysis system can be any of the systems described or otherwise envisioned herein. The emergency department workflow analysis system can be a single system or multiple different systems.

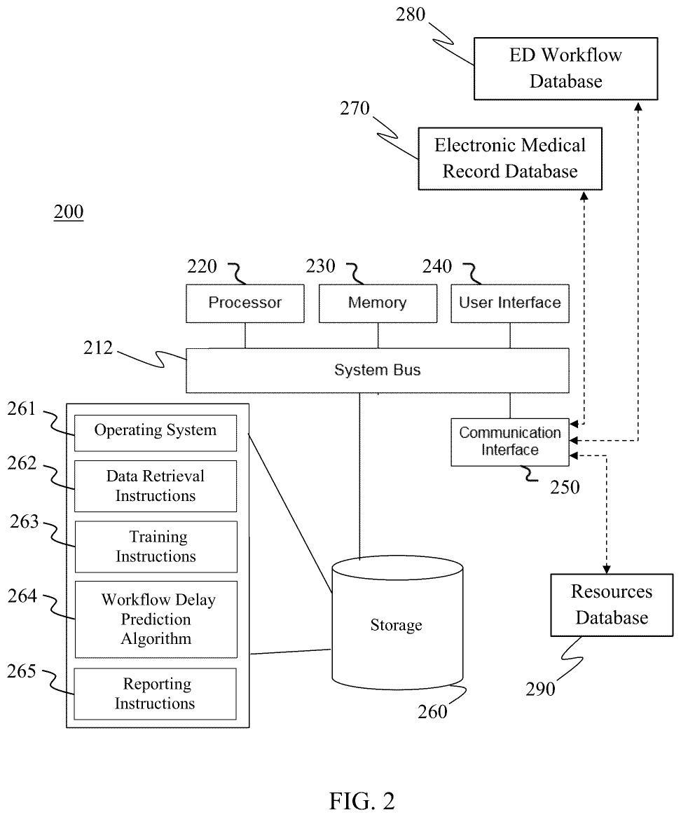

At step 110 of the method, an emergency department workflow analysis system is provided. Referring to an embodiment of emergency department workflow analysis system 200 as depicted in FIG. 2, for example, the system comprises one or more of a processor 220, memory 230, user interface 240, communications interface 250, and storage 260, interconnected via one or more system buses 212. It will be understood that FIG. 2 constitutes, in some respects, an abstraction and that the actual organization of the components of the system 200 may be different and more complex than illustrated. Additionally, ED workflow analysis system 200 can be any of the systems described or otherwise envisioned herein. Other elements and components of the ED workflow analysis system 200 are disclosed and/or envisioned elsewhere herein.

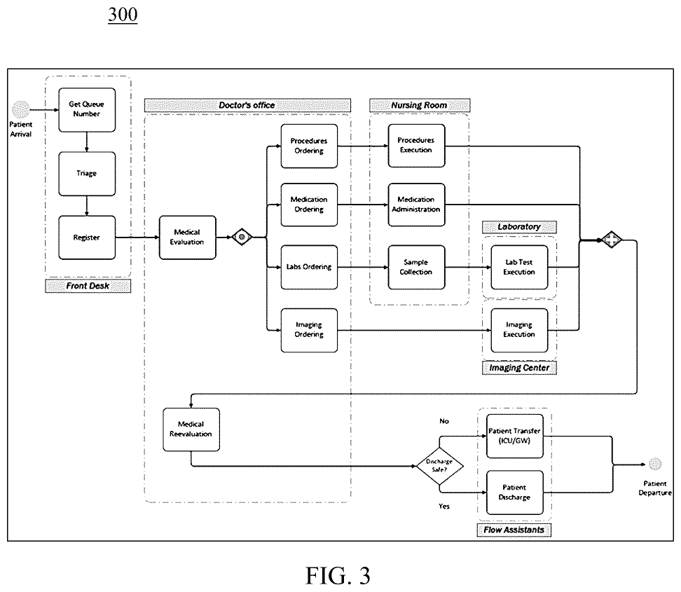

Referring to FIG. 3, in one embodiment, is a flowchart 300 of a general ED workflow. According to an embodiment, an ED workflow begins with the arrival of the patient at the ED, and the patient gets a queue number. After getting the queue number, the patient goes to triage room where vital signs are assessed, and a clinical priority is assigned to the patient. If the patient is classified as a high priority, they are directed to medical evaluation, otherwise, they go to the ED reception for registration. The next step is the medical evaluation, where the doctor assesses the patient and can order procedures, medications, and/or lab/imaging tests. When procedures or medications are prescribed, the patient goes to the procedure or medication rooms, respectively, to receive these procedures or medications. If lab tests are ordered, the sample is collected and forwarded to the hospital's laboratory, which will execute the tests and input their result in the EMR system upon completion. When imaging is ordered, patients are referred to the image sector for their tests. The results will be inputted in the EMR system after the radiologist's report. Once all lab/imaging testes, procedures, and medications are completed, the patient will be reassessed by the doctor, who will decide whether the patient should be hospitalized or discharged.

There are many alternative pathways in this workflow. For example, the patient may arrive in a severe clinical condition and be referred directly for medical evaluation or to the procedure room. It is also possible for the patient to be evaluated by the doctor and discharged directly, without do any lab/imaging tests. None of these path variations in the workflow interfere with the functioning of the systems and methods described or otherwise envisioned herein.

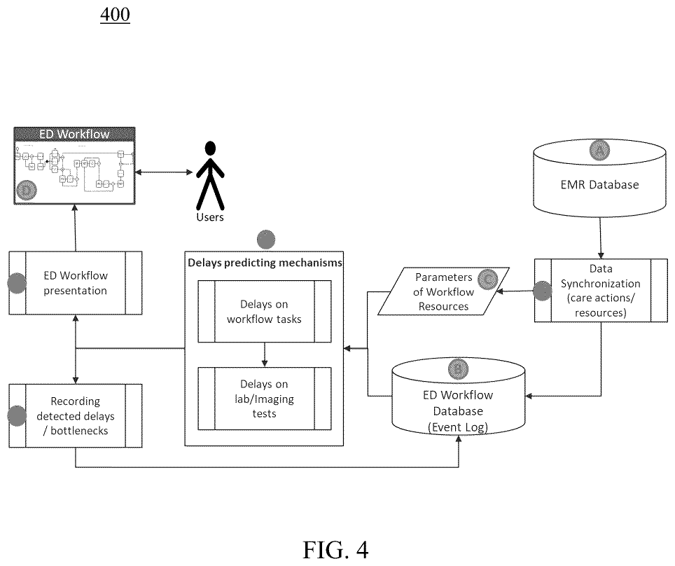

Referring to FIG. 4, in one embodiment, is a schematic representation 400 of a possible ED workflow analysis system 200, comprising four main components. The first component is responsible for populating the ED workflow database (B) from data stored in the electronic medical record (EMR), represented by item (A). The ED workflow database stores information related to the patient flow in the ED and the execution of workflow tasks.

According to an embodiment, the Data Synchronization component (1) identifies, from EMR data (A), the information regarding the execution of each task of the ED general workflow (D) and inserted them in the ED workflow database (B) following the format of an Event Log. The Data Synchronization component (1) is also responsible for loading the parameters file (C) regarding the available resources for execution of the workflow tasks. For this, it is necessary to read other databases beyond EMR. In the example of the ED workflow, it can include data such as professionals on duty, equipment maintenance, etc.

According to an embodiment, the second component of the system comprises the one or more algorithms aimed to predict delays in the ED workflow tasks. The algorithm(s) predicts delays for each task from workflow, and identifies individual patients with possibility of delays in the execution of their lab and imaging tests.

According to an embodiment, the results of the algorithm are used by the next two components of the system. One of the components (3) is responsible for presenting, in a graphic format, the status ED workflow, showing delays, bottlenecks and other important information for managing the workflow. The fourth component of the system (4) is responsible for recording the bottlenecks and delays in a specific table in the ED workflow database so that they can be analyzed in the future, with the purpose of improving the department's workflow.

At step 120 of the method, ED workflow analysis system 200 receives or obtains patient information about one or more patients in the ED. The patient data can be any information about the patient that the system can or may utilize for analysis as described or otherwise envisioned herein. According to an embodiment, the patient data comprises one or more of demographic information about the patient, medical history of the patient, a diagnosis for the patient, treatment and care for the patient, any labs, imaging, or other testing being done or planned for the patient, and any other information. For example, demographic information may comprise information about the patient such as name, age, body mass index (BMI), and any other demographic information. The medical history of the patient may be any historical admittance or discharge information, historical treatment information, historical diagnosis information, historical exam or imaging information, and/or any other information. The diagnosis for the patient may be any information about a medical diagnosis for the patient, historical and/or current. The treatment and care for the patient can be anything that is being done for the patient in the ED, or can be any care that is being utilized by the patient in the ED or any other medical setting or under any medical supervision. The labs, imaging, or other testing being done or planned for the patient can be any testing or imaging that the ED has prescribed, or is likely to prescribe, for the patient based on the patient's demographics, history, complaints, or diagnosis, for example.

The patient data is received from one or a plurality of different sources. According to an embodiment, the patient data is received from, retrieved from, or otherwise obtained from an electronic medical record (EMR) database or system 270. The EMR database or system may be local or remote. The EMR database or system may be a component of the ED workflow analysis system, or may be in local and/or remote communication with the ED workflow analysis system. The received patient data may be utilized immediately, or may be stored in local or remote storage for use in further steps of the method.

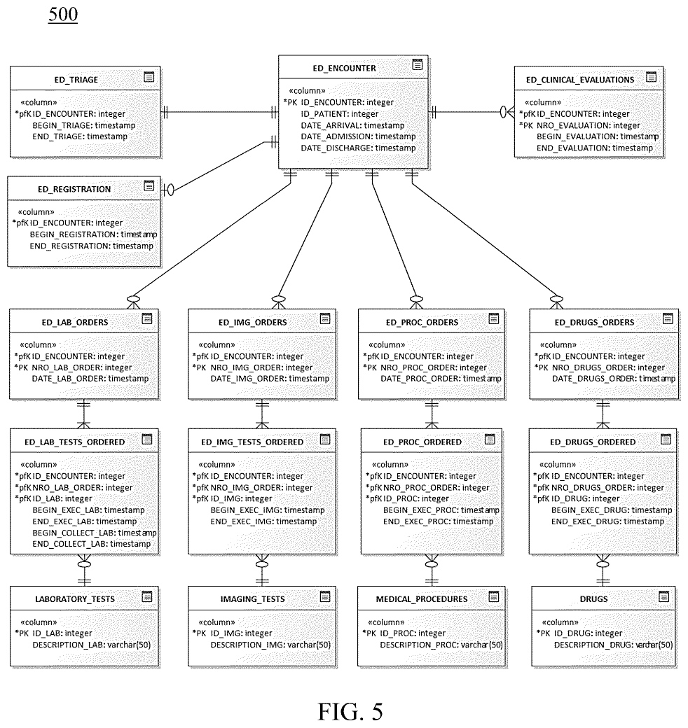

Referring to FIG. 5, in one embodiment, is a schematic representation 500 of patient data that is stored in the EMR database or system 270 and can be obtained or received by the ED workflow analysis system 200. A relational model, including the displayed tables and columns is utilized solely to facilitate understanding. It is not necessary that the data are stored in relational format or comprise all the information described in FIG. 5.

According to an embodiment, the EMR data utilized by ED workflow analysis system 200 refers to a patient's visit to the ED with the respective medical care provided to them. Considering that the focus of the system is the prediction of delays in the ED workflow, in accordance with one embodiment only the execution times of the ED activities will be needed.

According to an embodiment, the data subset can contain basic information regarding the visit to ED (ED_CONCOUNTER), such as encounter ID, patient ID, and the dates/times of the patient's arrival, discharge, or admission (if applicable). Each visit to the ED implies a triage (ED_TRIAGE), a registration at reception (ED_REGISTRATION) and at least one clinical evaluation (ED_CLINICAL_EVALUATION). During clinical evaluation, the doctor may order laboratory tests (ED_LAB_ORDERS), imaging tests (ED_IMG_ORDERS), medical procedures (ED_PROC_ORDERS) and administration of medications (ED_DRUGS_ORDERS). Each request type can contain one or more items (ED_LAB_TESTS_ORDERED, ED_IMG_TESTS_ORDERED, ED_PROC_ORDERED, ED_DRUGS_ORDERED). The patient information received by the system may comprise any of this, or other, information.

At step 130 of the method, the ED workflow analysis system 200 receives or obtains information from an ED workflow database 280. According to an embodiment, the information comprises patient flow information. The patient flow information may include, for example, data about patient flow in the ED including information about execution of one or more past, current, and/or future workflow tasks for the one or more patients in the ED. This patient flow information is one of the pieces of input information that the ED workflow analysis system will utilize to make an ED delay prediction as described or otherwise envisioned herein.

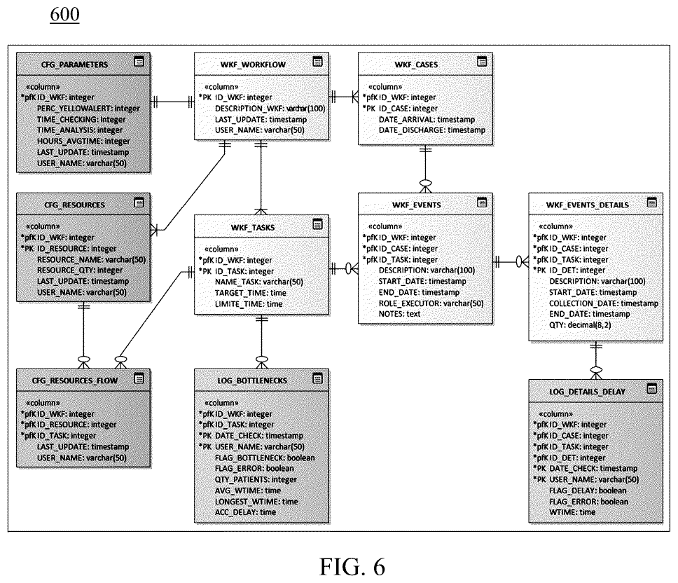

Referring to FIG. 6, in one embodiment, is a schematic representation 600 of an ED workflow database and representative data. According to an embodiment, the ED Workflow Database optionally stores one or more of three different types of data, as shown in FIG. 5.

According to an embodiment, the ED Workflow Database comprises data related to those retrieved from EMR, which provide information regarding the execution of the ED workflow tasks. These data are stored in the ED workflow database as an event log format, such as shown in the tables WKF_CASES, WKF_EVENTS, and WKF_EVENT_DETAILS. The table WKF CASES stores information regarding the patients (cases) who visited the ED. For the purposes of the tool, only the arrival and discharge date may be necessary. The table WKF EVENTS stores the event log for the tasks executed for each patient. This table comprises the start and end date of execution, beyond the description of the task, the role of executor and some additional notes. Some workflow tasks can comprise sub-tasks, for example, lab tests. One lab test order can include several lab tests, and each one of them has its execution time. Thus, the table WKF EVENT DETAILS is used to store information regarding the sub-tasks.

According to an embodiment, the ED Workflow Database also comprises one or more parameters required for functioning of the tool, as described in greater detail below. According to an embodiment, three database table stores these parameters. The table CFG_PARAMETERS stores the auxiliary parameters related to the presentation of results and the log of delays, including “% for Yellow alert,” “Time for checking,” “Time for analysis,” and “Hours for waiting time.” The tables CFG_RESOURCES and CFG_RESOURCES_FLOW store the parameters related to the resources used in the task execution with their respective quantities.

According to an embodiment, the ED Workflow Database also comprises log files regarding the delays and bottlenecks detected by the tool. According to an embodiment, two database tables are used to do it: LOG_BOTTLENECKS and LOG_DETAILS_DELAY.

According to an embodiment, the patient flow information is received from one or a plurality of different sources. According to an embodiment, the patient flow information is received from, retrieved from, or otherwise obtained from an ED workflow database 280. The ED workflow database may be local or remote. The ED workflow database may be a component of the ED workflow analysis system, or may be in local and/or remote communication with the ED workflow analysis system. The received patient flow information may be utilized immediately, or may be stored in local or remote storage for use in further steps of the method.

According to an embodiment, the information received by the ED workflow analysis system 200 from the ED workflow database 280 further comprises one or more parameters for each of resources utilized in workflow task execution and target time for workflow task execution. The ED workflow analysis system may require, for example, one or more parameters to calibrate its algorithms. According to an embodiment, there are two types of parameters; those related to the resources needed to the execution of the tasks, and those related to the target times for each task.

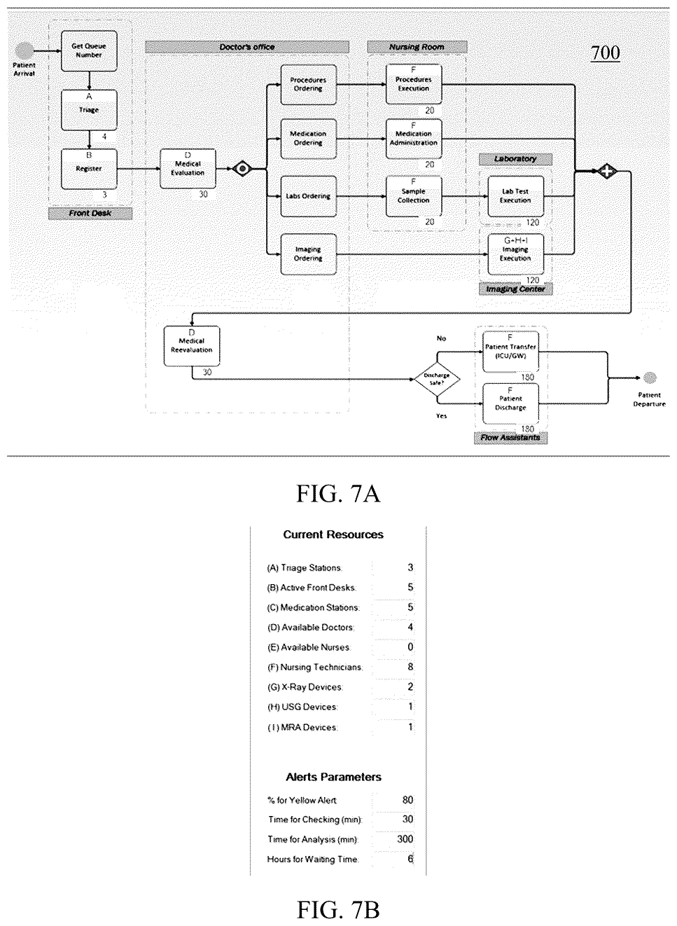

Referring to FIGS. 7A and 7B, in one embodiment, are a possible interface for setting one or more parameters of the system. Depicted in FIG. 7B are parameters related to the resources used in the tasks execution with their respective quantities. Depicted in FIG. 7A is a workflow graph in which the user includes the target time (below the box) and the resources used (inside the box) for each task. For example, as shown in FIG. 7A, the “Medical Evaluation” task uses the resource D (“Available Doctors”) and the target time for executing this task is 30 minutes.

The values of the parameters related to task resources can be automatically updated by the synchronization component when they are available in EMR or other digital media; otherwise, they can be manually added by the user whenever there is a change in the quantity of these resources.

At the bottom left of FIG. 7A are the auxiliary parameters related to the presentation of results and the log of delays. According to an embodiment, the “% for Yellow alert” parameter defines when an attention alert should be signaled to the user. For example, if the value 80 was defined for this parameter, a signal will be sent on the screen if 80% of the task's target time has already elapsed. The “Time for checking” parameter defines the time interval in which the ED workflow status and the respective delays will be automatically recorded in the log file. This parameter is also used by sync component to check and update the parameters related to task resources automatically. The “Time for analysis” parameter defines the time in which the user will receive a message to analyze the delays and bottlenecks detected by the tool. The “Hours for waiting time” parameter defines how many hours the algorithms must consider to calculating the average waiting time. The example in FIG. 7A indicates that the average waiting time for the task will be calculated considering the patients (encounters) from the last six hours.

According to an embodiment, the one or more parameters are received from one or a plurality of different sources. According to an embodiment, the one or more parameters are received from, retrieved from, or otherwise obtained from an ED workflow database 280. The ED workflow database may be local or remote. The ED workflow database may be a component of the ED workflow analysis system, or may be in local and/or remote communication with the ED workflow analysis system. The received one or more parameters may be utilized immediately, or may be stored in local or remote storage for use in further steps of the method.

At step 140 of the method, ED workflow analysis system 200 receives or obtains information from a resources database 290. According to an embodiment, the information comprises resource information comprising data about resources for the ED. The resource information may be any information about resources within the ED or care facility that may impact the ED workflow and/or patient care. For example, the resource information may comprise information about staffing, workload, device (e.g., imaging devices) status, and more. According to an embodiment, the resource information may be contained within the resource parameters discussed above, and thus step 140 may be contained within step 130.

The resource information can be received from one or a plurality of different sources. According to an embodiment, the resource information is received from, retrieved from, or otherwise obtained from the ED workflow database 280 and/or a resources database 290. The database may be local or remote. The database may be a component of the ED workflow analysis system, or may be in local and/or remote communication with the ED workflow analysis system. The received resource information may be utilized immediately, or may be stored in local or remote storage for use in further steps of the method.

At step 150 of the method, the ED workflow analysis system 200 processes the received patient information, received patient flow information, received one or more parameters, and received resource information. According to an embodiment, a trained workflow delay prediction algorithm of the ED workflow analysis system processes this received or obtained information in order to generate a predicted delay in one or more of the current or future workflow tasks for the one or more patients, wherein the generated predicted delay is a department-level delay or a patient-level delay.

The trained workflow delay prediction algorithm can be any machine learning algorithm, neural network, classifier, or other algorithm capable of utilizing the described input to generate the described output. The trained model of the ED workflow analysis system generates, using the received input data, a predicted delay in one or more of the current or future workflow tasks for the one or more patients.

According to an embodiment, at optional step 152 of the method, the trained workflow delay prediction algorithm of the ED workflow analysis system processes the received or obtained information, and optionally the generated predicted delay, to generate one or more recommendations for preventing or alleviating the generated predicted delay. For example, since the system is informed about where the predicted delay may be and/or what is causing the predicted delay, the system can then analyze the predicted delay to determine what one or more actions may prevent the predicted delay, and/or alleviate the impact of that predicted delay. For example, the trained workflow delay prediction algorithm may determine, in response to predicting a delay, that reallocating human resources from one patient or department to another may prevent a predicted workflow delay. As another example, the trained workflow delay prediction algorithm may determine, in response to predicting a delay, that increasing resources in a testing laboratory or imaging department may prevent or alleviate a predicted delay.

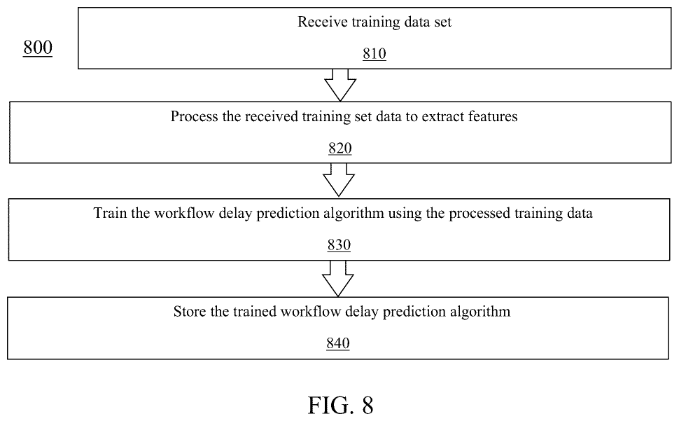

Referring to FIG. 8, in one embodiment, is a flowchart of a method 800 for training the workflow delay prediction algorithm of the ED workflow analysis system 200. At step 810 of the method, the system receives a training data set comprising ED workflow data, such as historical ED workflow data about the flow of patients in the ED, resources utilized, and more. The training data can comprise any information or data necessary to train the workflow delay prediction algorithm. The training data may be stored in and/or received from one or more databases. The database may be a local and/or remote database. For example, the ED workflow analysis system may comprise a database of training data.

According to an embodiment, the ED workflow analysis system may comprise a data pre-processor or similar component or algorithm configured to process the received training data. For example, the data pre-processor analyzes the training data to remove noise, bias, errors, and other potential issues. The data pre-processor may also analyze the input data to remove low quality data. Many other forms of data pre-processing or data point identification and/or extraction are possible.

At step 820 of the method, the system processes the received information to extract ED workflow features. The ED workflow features may be any features which will be utilized to train the workflow delay prediction algorithm, such as historical ED workflow, patient data, resource data, parameter data, and/or any other data. Feature extraction can be accomplished by a variety of embodiments for feature identification, extraction, and/or processing, including any method for extracting features from a dataset. The outcome of a feature processing step or module of the system is a set of ED workflow features about the ED, which thus comprises a training data set that can be utilized to train the classifier.

At step 830 of the method, the system trains the machine learning algorithm, which will be the algorithm utilized in analyzing ED information as described or otherwise envisioned. The machine learning algorithm is trained using the extracted features according to known methods for training a machine learning algorithm. According to an embodiment, the algorithm is trained, using the processed training dataset, to generate a prediction of ED workflow delay, as described or otherwise envisioned herein. A generated report can comprise any of the information described or otherwise envisioned herein, including but not limited to the predicted ED workflow delay, a workflow recommendation, information about the causes of the predicted ED workflow delay, and/or other information.

At step 840 of the method, the trained workflow delay prediction algorithm is stored for future use. According to an embodiment, the trained model may be stored in local or remote storage.

According to an embodiment, the ED workflow analysis system may comprise a data synchronization component, such as that shown in FIG. 3, in order to facilitate analysis by the trained algorithm. According to an embodiment, the Data Synchronization component comprises a mechanism that executes SQL statements, implemented by database stored procedures, to retrieve EMR data and load them into ED Dashboard database. These stored procedures are scheduled to run automatically in a period defined by the user. The basic principle of this component is to use one SQL statement (stored procedure) for each task of ED workflow. This principle aims ensuring the software engineering good practices of cohesion and coupling, and consequently facilitate the maintenance and evolution of the system.

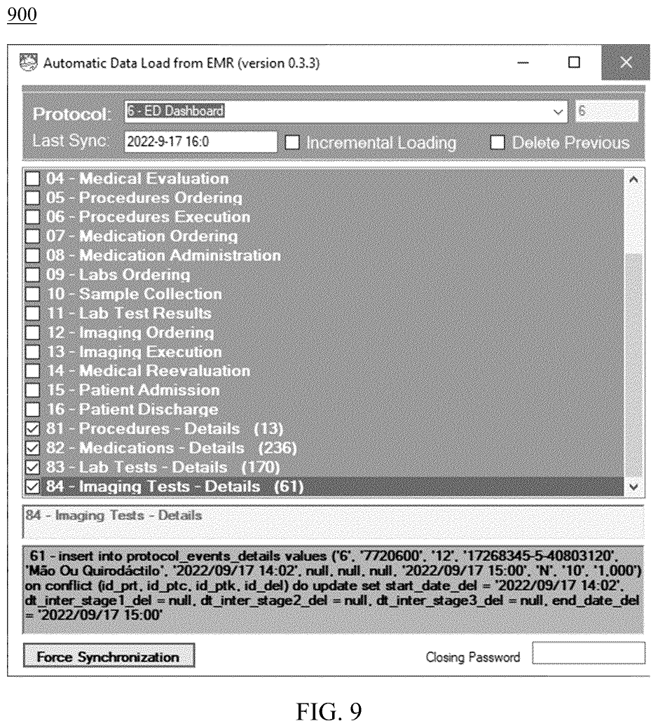

Referring to FIG. 9, in accordance with an embodiment, is an interface 900 for a data synchronization component. In the center of the screen is a scheduling area, comprising one row for each ED workflow task. Each line contains a SQL command that is scheduled to be running according to the synchronization time parameter. In the bottom of screen, there is the SQL command executed for the “Imaging Test—Details” task.

According to an embodiment, the ED workflow analysis system may comprise a delay predicting component, such as that shown in FIG. 3 and discussed above, in order to generate the predicted ED workflow delay and/or workflow recommendations. According to an embodiment, the delay predicting component comprises the trained workflow delay prediction algorithm that is configured to predict delays for each task from workflow, in a dept level, and identify individual patients with possibility of delays in the execution of their lab and imaging tests.

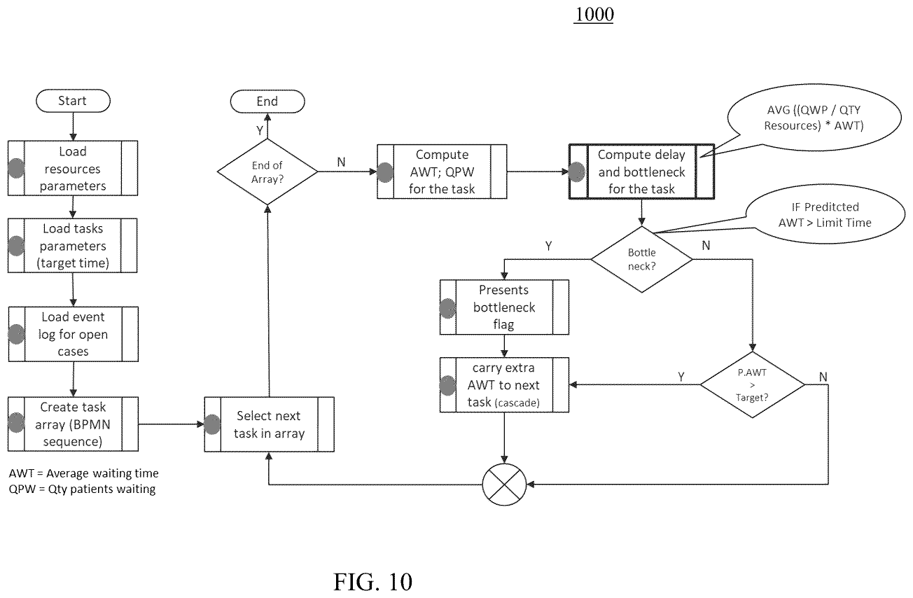

Referring to FIG. 10, in one embodiment, is a schematic flowchart 1000 of the functionality of the trained workflow delay prediction algorithm. In accordance with an embodiment, the first three steps consist of retrieving the input information requested by the algorithm: the parameters of workflow tasks (2); the resources available to tasks execution (1); and information regarding the open cases (3) running in the ED workflow (patients that are in the ED).

The next step (4) is to create an array to store the parameters to each workflow task. This array has one position for each task and each position comprise six variables: task ID, subsequent task ID, execution target time, quantity of available resources; quantity of patients waiting for the task (QPW), and the average waiting time (AWT) computed according to the parameter “Hours for waiting time.” The first four variables are retrieved from the input data (ED workflow database), while the last two are calculated by the algorithm (in the next steps).

After the creation of the tasks array, a loop is started to evaluate each task (5); in which the AWT and QWP (6) are calculated, the estimation of delays and bottlenecks is evaluated (7), the bottleneck flag is activated (8), and the extra AWT is accumulated in the subsequent task (9).

The QPW of a task consists of the sum of all running instances (e.g., patients who are in the ED) that have already concluded the previous tasks and have not yet enter the current task or in the subsequent ones. The task's AWT is the average waiting time of the patients for that task, considering those patients which have already completed this task in the last X hours, where X is the value assigned to the parameter “Hours for waiting time.”

A bottleneck in a task is detected when the estimated average waiting time for the instances that are waiting is greater than the task's target time. The estimated waiting time considers the volume of resources available and the number of instances waiting. Estimated waiting time can be calculated by the formula: AVG((QWP/Qty Resources)*AWT).

Consequently, the amount of delay predicted for that task is: ((estimated waiting time−target time)*QWP). When a bottleneck is identified, the respective flag in the task array receives the value true, and the extra AWT, which means the estimated waiting time minus target time, is accumulated in the AWT of the subsequent task.

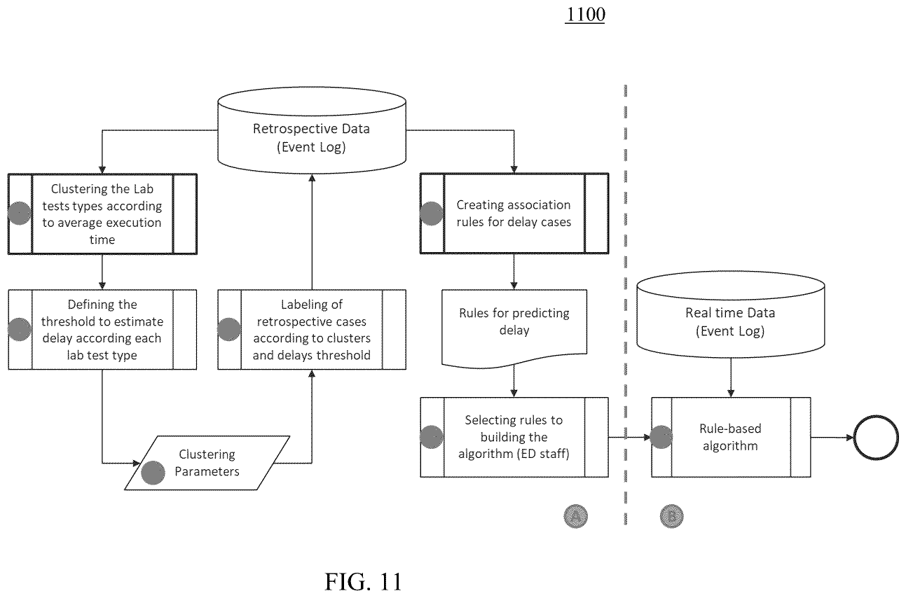

Referring to FIG. 11, in one embodiment, is a schematic flowchart 1100 of the prediction of delays in ED workflow caused by lab and imaging tests. The algorithm is represented by item 7 in FIG. 11. All other components of the schematic are intermediate steps to discover a set of rules that can be used to build this algorithm.

The proposed method contains two distinct groups of activities. The first (A) is intended to discover the association rules that will be used to implement the rule-based algorithm (B) that is a part of the tool proposed in this invention. The first group of activities is based on machine learning techniques and uses a retrospective dataset. First step is clustering the different types of lab/imaging tests according to the average time for their execution (1). Once created the clusters, a threshold (2) for each cluster must be defined to establish a criterion (3) for classifying delays. Once the criteria for delays have been established (4), a retrospective dataset, which will be used for training of association rules, must be classified according to this criterion. Having a dataset properly classified, the association rules techniques are applied (5) and a set of association rules will be produced. These rules associate some contextual variables to the occurrence of delays in the lab/imaging test execution. All discovered association rules are analyzed by experts (6) and those selected by them will be used to implement the rule-based algorithm (7).

According to an embodiment, several techniques should be applied to select the best algorithm to generate the most cohesive clusters. In one embodiment, testing determined that K-means presented the best outcomes for a particular ED. In other EDs and other cases, other algorithms may perform better.

According to an embodiment, there may be a threshold to estimate or determine or predict a delay. The definition of the threshold can be done together to end users (ED manager), as this threshold need to make sense to them. A good criterion for threshold definition, if the clusters are well cohesive, is to adopt a percentage derived from the ration between standard deviation and mean. In one example, the average time to execute a test from cluster one is 99.17 minutes, with a standard deviation of 43.38 minutes. Thus, the ration between them is 0.43, which should be used as the threshold for the cluster. In this way, if the execution time for any test belong to this cluster is 43% higher than the average time of the cluster, then the test will be classified as delayed.

According to an embodiment, after defining the thresholds, a table with the cluster parameters can be generated, which will be used to classify the retrospective dataset (that will be used for training the association rules). This table needs to have basically four attributes: the test identifier, its average execution time, the identifier of the cluster it belongs to, and the threshold.

According to an embodiment, the system can label retrospective cases. The training dataset used to discover association rules must include the predictor attribute (flag delay) and any contextual variables that can have some correlation with it. By using the threshold stored in the clustering parameters table, all records from training dataset must be classified, assigning the value “Y” to predictor attribute in case of delays, otherwise, assigning the value “N.”

According to an embodiment, the system can discover and select association rules. As with clustering, the method does not define a specific machine learning technique for discovering association rules. Several techniques should be applied to select the one that generate the most coherent association rules. In one example, the Apriori algorithm presented the best result, but other algorithms may be better in other situations. It may be important to define a minimum confident rate to limit the quantity of produced rules and to ensure more strong rules. The association rules produced can be organized and discussed with end users to select all one which will be used to implement the rule-based algorithm. In addition to rules discovered by machine learning techniques, it is possible to add other business rules. In one example, two business rules were included.

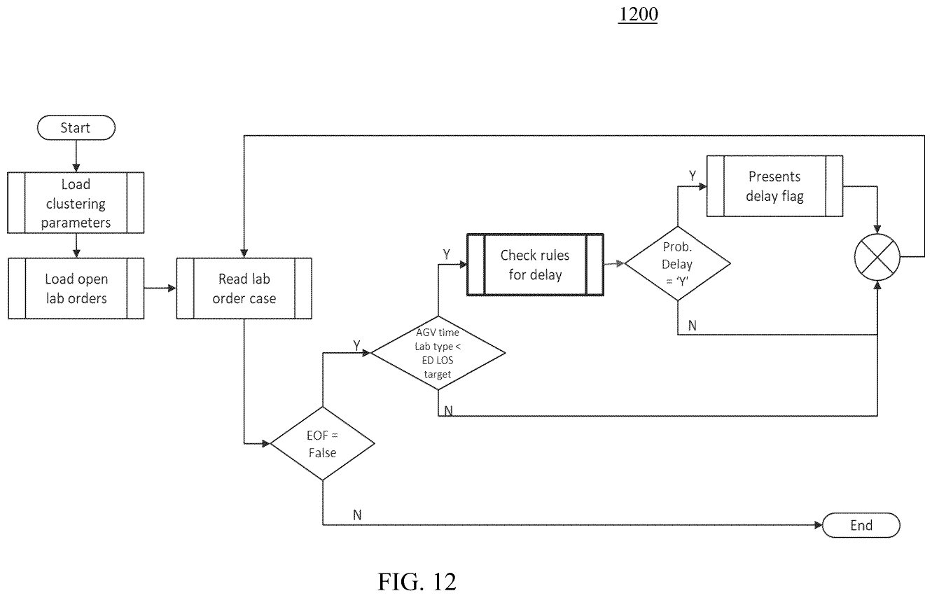

According to an embodiment, the selected association rules are implemented to the rule-based algorithm through if-then-else clauses. It is recommended that the association rules model be periodically retrained to find new rules that can be included in the rule-based algorithm. Referring to FIG. 12, in one embodiment, is a potential scheme for the rule-based algorithm, which runs in real time and analyses all open imaging and lab tests orders. The rules embedded in the algorithm are applied for each test included in the lab/imaging order. If the contextual variables fit within any of the rules, the flag of delay is activated. The component 3 of the tool—referring back to FIG. 3—is responsible for presenting to users the lab/imaging orders classified as potential delay.

Returning to method 100 depicted in FIG. 1, at step 160 of the method, in accordance with an embodiment, the generated predicted delay in one or more of the current or future workflow tasks for the one or more patients, and optionally the one or more recommendations, are provided via a user interface of the ED workflow analysis system. The generated predicted delay and/or recommendations may be provided to a user, which can be a medical professional, and/or to any other individual authorized to see or review the information. The generated predicted delay and/or recommendations can be provided to a user via any user interface or other information communication system. For example, the information can be transmitted or otherwise communicated via wired and/or wireless communication to a local or remote viewer, such as a remote medical professional.

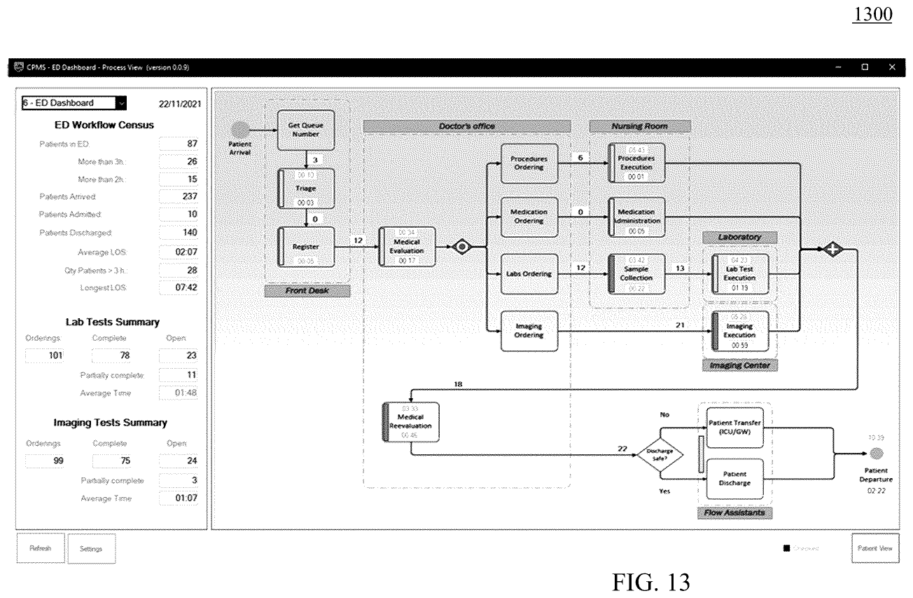

Referring to FIG. 13, in one embodiment, is a possible embodiment of a graphical display of the generated predicted ED workflow delay. According to an embodiment, one goal of the display is to provide to clinical team and to ED managers a simple user interface for presenting the status of patient flow in the ED, showing the detected delays and bottlenecks. This component offers two distinct views: department and patients. In the department view, ED managers can have a real-time overview of the workflow tasks in execution and the flow of patients in each one of them. In the patient view, managers can see more details about the patients in the ED, such as length of stay, open and completed tests, procedures done, etc.

FIG. 13 is a department view. The left part of the screen summarizes important KPIs, which provide the manager with an overview on how ED is running since the beginning of the day. It shows the number of patients who are in the ED in that that time, as well as the number of patients who have been through the ED and were either discharged or admitted. Three others significant KPIs are the average length of stay, the number of patients who exceeded three hours in the ED (target time) and the longest length of stay. Also presented are some KPIs related to lab and imaging tests, such as the number of open and completed orders, as well as the average execution time. These additional KPIs help the users understand the context of the delays and bottlenecks detected by the tool algorithms.

On the right side of the screen, there is the ED workflow at any given moment. Information regarding patient flow is presented in a graphical and dynamic format, which is refreshed every minute. Upper arrows show the number of patients that are waiting for that task. Inside the box (which represents a task) two important KPIs are presented. The value under the task name indicates the current waiting time for the task, considering the parameter “Hours for waiting time.” The value above the task name indicates the longest wait time for that task. That is, the waiting time of that patient who has the longest wait time.

When any of these values exceeds the target time, it is displayed in red color or some other method to indicate the target time is exceeded. Otherwise, when it exceeds the value of parameter “% for Yellow Alert”, it is displayed in yellow color or some other method to so indicate. When a bottleneck is detected, the sidebar of the box representing the task changes its color from green to red.

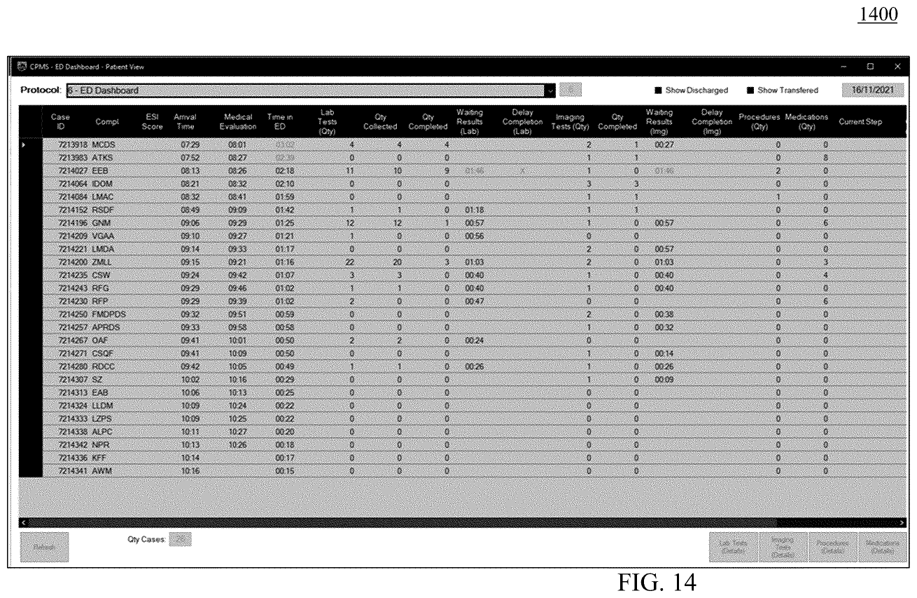

Referring to FIG. 14 is the patient view of the provided display, in accordance with one embodiment. This display, which can be in tabular format, presents some important information about patients who are in the ED at that time. This allows ED managers to early identify cases that are running out of schedule.

For each patient, the following information is presented: arrival time at the ED, medical evaluation time, length of stay, lab and imaging open orders, waiting time for test results, and a column that indicates the probability of delays in these tests. The probability of exam delays is indicated only by a flag (X). Columns showing patient length of stay and waiting time for test results change to red color when that time exceeds the target time; and change to yellow color when it exceeds the value of parameter “% for Yellow Alert.” In the bottom right-hand corner, there are four buttons that trigger more details about the patient: laboratory tests, imaging tests, procedures, and medications. A details screen can expand all items contained in the orders. The purpose of the details screen is to help managers both in understanding the reasons for delays and in the mitigation of the problem. They can take actions on the item which is causing the issue.

Returning to method 100 in FIG. 1, at step 170 of method 100, the generated predicted workflow delay is stored in a database for subsequent analysis by the system, including but not limited to training or re-training the workflow delay analysis algorithm. The generated predicted workflow delay may be stored in any database. The database may be a local or remote database and is in direct and/or indirect communication with system 200.

In accordance with an embodiment, the generated predicted workflow delay is stored in a log file, including the bottlenecks and delays detected. The component is activated automatically according to parameter “Time for Checking” and registers which tasks are in bottleneck, the average execution time, the longest waiting time, and the number of patients waiting for the task. Likewise, lab and imaging tests flagged as potential delays are recorded in that log file. This log file can be used for retrospective analysis aiming a better understanding regarding bottlenecks and delays, as well as, providing insights for improving the ED workflow.

Referring to FIG. 2 is a schematic representation of an emergency department workflow analysis system 200. System 200 may be any of the systems described or otherwise envisioned herein, and may comprise any of the components described or otherwise envisioned herein. It will be understood that FIG. 2 constitutes, in some respects, an abstraction and that the actual organization of the components of the system 200 may be different and more complex than illustrated.

According to an embodiment, system 200 comprises a processor 220 capable of executing instructions stored in memory 230 or storage 260 or otherwise processing data to, for example, perform one or more steps of the method. Processor 220 may be formed of one or multiple modules. Processor 220 may take any suitable form, including but not limited to a microprocessor, microcontroller, multiple microcontrollers, circuitry, field programmable gate array (FPGA), application-specific integrated circuit (ASIC), a single processor, or plural processors.

Memory 230 can take any suitable form, including a non-volatile memory and/or RAM. The memory 230 may include various memories such as, for example L1, L2, or L3 cache or system memory. As such, the memory 230 may include static random access memory (SRAM), dynamic RAM (DRAM), flash memory, read only memory (ROM), or other similar memory devices. The memory can store, among other things, an operating system. The RAM is used by the processor for the temporary storage of data. According to an embodiment, an operating system may contain code which, when executed by the processor, controls operation of one or more components of system 200. It will be apparent that, in embodiments where the processor implements one or more of the functions described herein in hardware, the software described as corresponding to such functionality in other embodiments may be omitted.

User interface 240 may include one or more devices for enabling communication with a user. The user interface can be any device or system that allows information to be conveyed and/or received, and may include a display, a mouse, and/or a keyboard for receiving user commands. In some embodiments, user interface 240 may include a command line interface or graphical user interface that may be presented to a remote terminal via communication interface 250. The user interface may be located with one or more other components of the system, or may located remote from the system and in communication via a wired and/or wireless communications network.

Communication interface 250 may include one or more devices for enabling communication with other hardware devices. For example, communication interface 250 may include a network interface card (NIC) configured to communicate according to the Ethernet protocol. Additionally, communication interface 250 may implement a TCP/IP stack for communication according to the TCP/IP protocols. Various alternative or additional hardware or configurations for communication interface 250 will be apparent.

Storage 260 may include one or more machine-readable storage media such as read-only memory (ROM), random-access memory (RAM), magnetic disk storage media, optical storage media, flash-memory devices, or similar storage media. In various embodiments, storage 260 may store instructions for execution by processor 220 or data upon which processor 220 may operate. For example, storage 260 may store an operating system 261 for controlling various operations of system 200.

It will be apparent that various information described as stored in storage 260 may be additionally or alternatively stored in memory 230. In this respect, memory 230 may also be considered to constitute a storage device and storage 260 may be considered a memory. Various other arrangements will be apparent. Further, memory 230 and storage 260 may both be considered to be non-transitory machine-readable media. As used herein, the term non-transitory will be understood to exclude transitory signals but to include all forms of storage, including both volatile and non-volatile memories.

While system 200 is shown as including one of each described component, the various components may be duplicated in various embodiments. For example, processor 220 may include multiple microprocessors that are configured to independently execute the methods described herein or are configured to perform steps or subroutines of the methods described herein such that the multiple processors cooperate to achieve the functionality described herein. Further, where one or more components of system 200 is implemented in a cloud computing system, the various hardware components may belong to separate physical systems. For example, processor 220 may include a first processor in a first server and a second processor in a second server. Many other variations and configurations are possible.

According to an embodiment, the electronic medical record system 270 is an electronic medical records database from which the information about patients, including demographic, diagnosis, and/or treatment information, may be obtained or received. According to an embodiment, the electronic medical record system 270 is an electronic medical records database from which the training data utilized to train the workflow delay prediction algorithm. The training data can be any data that will be utilized to train the algorithm. The electronic medical records database may be a local or remote database and is in direct and/or indirect communication with system 200. Thus, according to an embodiment, the system comprises an electronic medical record database or system 270.

According to an embodiment, the system comprises an ED workflow database 280 comprising patient flow information. The patient flow information may include, for example, data about patient flow in the ED including information about execution of one or more past, current, and/or future workflow tasks for the one or more patients in the ED. This patient flow information is one of the pieces of input information that the ED workflow analysis system will utilize to make an ED delay prediction as described or otherwise envisioned herein. The ED workflow database may be a local or remote database and is in direct and/or indirect communication with system 200. Thus, according to an embodiment, the system comprises an ED workflow database 280.

According to an embodiment, the system comprises a resources database 290 comprising resource information with data about resources for the ED. The resource information may be any information about resources within the ED or care facility that may impact the ED workflow and/or patient care. For example, the resource information may comprise information about staffing, workload, device (e.g., imaging devices) status, and more. The resources database may be a local or remote database and is in direct and/or indirect communication with system 200. Thus, according to an embodiment, the system comprises a resources database 290.

According to an embodiment, storage 260 of system 200 may store one or more algorithms, modules, and/or instructions to carry out one or more functions or steps of the methods described or otherwise envisioned herein. For example, the system may comprise, among other instructions or data, data retrieval instructions 262, training instructions 263, a trained workflow delay prediction algorithm 264, and/or reporting instructions 265.

According to an embodiment, data retrieval instructions 262 direct the system to retrieve, receive, or otherwise obtain information from one or more sources, which the system will utilize to generate a delay prediction. For example, the data retrieval instructions 262 direct the system to retrieve, receive, or otherwise obtain patient information about one or more patients in the ED from an EMR database or system 270, patient flow information from an ED workflow database 280, and/or resource information from a resources database 290. Once retrieved, this information may be utilized immediately or stored in a local and/or remote database for subsequent use.

According to an embodiment, training instructions 263 direct the system to train a workflow delay prediction algorithm, in order to generate the trained workflow delay prediction algorithm 264. Referring to FIG. 8, in one embodiment, is a flowchart of a method 800 for training the workflow delay prediction algorithm of the ED workflow analysis system 200, which may be accomplished by the training instructions 263. Thus, the system comprises a trained workflow delay prediction algorithm 264 configured to generate an ED delay prediction, as well as optionally one or more recommendations for preventing or alleviating that delay.

According to an embodiment, reporting instructions 265 direct the system to generate and provide to a user via a user interface information comprising the generated predicted ED workflow delay, as well as optionally one or more recommendations for preventing or alleviating that delay. Alternatively, the information may be communicated by wired and/or wireless communication to another device. For example, the system may communicate the information to a mobile phone, computer, laptop, wearable device, and/or any other device configured to allow display and/or other communication of the information.

According to an embodiment, the emergency department workflow analysis system is configured to process many thousands or millions of datapoints in the input data used to train the workflow delay prediction algorithm 264. For example, generating a functional and skilled trained workflow delay prediction algorithm using an automated process such as feature identification and extraction and subsequent training requires processing of millions of datapoints from input data and the generated features. This can require millions or billions of calculations to generate a novel trained workflow delay prediction algorithm from those millions of datapoints and millions or billions of calculations. As a result, each trained workflow delay prediction algorithm is novel and distinct based on the input data and parameters of the machine learning algorithm, and thus improves the functioning of the ED workflow analysis system. Thus, generating a functional and skilled trained workflow delay prediction algorithm comprises a process with a volume of calculation and analysis that a human brain cannot accomplish in a lifetime, or multiple lifetimes.

Additionally, the trained workflow delay prediction algorithm is configured to process many thousands or millions of datapoints as input, including but not limited to the received patient information about one or more patients in the ED from an EMR database or system 270, patient flow information from an ED workflow database 280, and/or resource information from a resources database 290, all utilized in real-time to generate an output, namely a predicted ED workflow delay and optionally one or more recommendations for preventing or alleviating that predicted ED workflow delay. In addition, the system can be configured to continually receive the described input data, perform the analysis, and provide periodic or continual ED workflow delay updates via the information provided to a user via the user interface. This requires the analysis of thousands or millions of datapoints on a continual basis to optimize the reporting, requiring a volume of calculation and analysis that a human brain cannot accomplish in a lifetime.

By providing an improved ED workflow analysis and ED workflow delay predictions, this novel ED workflow analysis system has an enormous positive effect on patient management and care compared to prior art systems. As just one example in a clinical setting, by providing a system that can provide improved ED workflow delay predictions, the system can facilitate and improve treatment, thereby improving patient care, increasing efficiency, and reduce costs.

All definitions, as defined and used herein, should be understood to control over dictionary definitions, definitions in documents incorporated by reference, and/or ordinary meanings of the defined terms.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.”

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified.

It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively.

While several inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize, or be able to ascertain using no more than routine experimentation, many equivalents to the specific inventive embodiments described herein. It is, therefore, to be understood that the foregoing embodiments are presented by way of example only and that, within the scope of the appended claims and equivalents thereto, inventive embodiments may be practiced otherwise than as specifically described and claimed. Inventive embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

What is claimed is:

1. A method for providing a delay prediction for emergency department (ED) workflow, comprising:
receiving, from an electronic medical record database, patient information about one or more patients in the ED;
receiving, from an ED workflow database, (i) patient flow information comprising data about patient flow in the ED including information about execution of one or more past, current, and/or future workflow tasks for the one or more patients in the ED and (ii) one or more parameters for both resources utilized in workflow task execution and target time for workflow task execution;
receiving, from a resources database, resource information comprising data about resources for the ED;
analyzing, with a trained workflow delay prediction algorithm, the received patient information, received patient flow information, received one or more parameters, and received resource information, to generate a predicted delay in one or more of the current or future workflow tasks for the one or more patients, wherein the generated predicted delay is a department-level delay or a patient-level delay; and
providing, via a graphical user interface, the generated predicted delay.

2. The method of claim 1, further comprising the steps of:
generating, by the trained workflow delay prediction algorithm, one or more recommendations for preventing or alleviating the generated predicted delay;
providing, via the graphical user interface, the one or more recommendations; and
administering the one or more recommendations to prevent or alleviate the generated predicted delay.

3. The method of claim 1, further comprising the step of storing, in a database, the generated predicted delay for subsequent analysis.

4. The method of claim 1, wherein the one or more parameters for the resources utilized in workflow task execution are predetermined by a user.

5. The method of claim 1, wherein the one or more parameters for the target time for workflow task execution are predetermined by a user.

6. The method of claim 1, wherein the provided generated predicted delay further comprises a graphic representation of workflow within the ED, and wherein the generated predicted delay is overlayed on the graphic representation at a location of the predicted delay within the workflow.

7. The method of claim 6, wherein the overlay of the generated predicted delay on the graphic representation comprises a color indicating the generated predicted delay.

8. The method of claim 1, wherein the provided generated predicted delay further comprises a graphic representation of each of a plurality of patients in the ED, and further comprises an indication to which of the plurality of patients in the ED the generated predicted delay applies.

9. A method for providing a delay prediction for emergency department (ED) workflow, comprising:
receiving, via a graphical user interface, a generated predicted delay in ED workflow and one or more recommendations for preventing or alleviating the generated predicted delay in ED workflow, wherein the predicted delay in ED workflow and one or more recommendations are generated by:
receiving, from an electronic medical record database, patient information about one or more patients in the ED;
receiving, from an ED workflow database, (i) patient flow information comprising data about patient flow in the ED including information about execution of one or more past, current, and/or future workflow tasks for the one or more patients in the ED and (ii) one or more parameters for both resources utilized in workflow task execution and target time for workflow task execution;
receiving, from a resources database, resource information comprising data about resources for the ED;
analyzing, with a trained workflow delay prediction algorithm, the received patient information, received patient flow information, received one or more parameters, and received resource information, to generate (i) a predicted delay in ED workflow and (ii) one or more recommendations for preventing or alleviating the generated predicted delay in ED workflow; and
administering the one or more recommendations to prevent or alleviate the generated predicted delay in ED workflow.

10. A system for providing a delay prediction for emergency department (ED) workflow, comprising:
an electronic medical record database comprising patient information about one or more patients in the ED;
an ED workflow database comprising: (i) patient flow information comprising data about patient flow in the ED including information about execution of one or more past, current, and/or future workflow tasks for the one or more patients in the ED and (ii) one or more parameters for both resources utilized in workflow task execution and target time for workflow task execution;
a resources database comprising resource information comprising data about resources for the ED;
a trained workflow delay prediction algorithm;
a processor configured to: (i) receive patient information, patient flow information, one or more parameters, and resource information; (ii) analyze, using the trained workflow delay prediction algorithm, the received patient information, received patient flow information, received one or more parameters, and received resource information, to generate a predicted delay in one or more of the current or future workflow tasks for the one or more patients, wherein the generated predicted delay is a department-level delay or a patient-level delay; and
a user interface configured to provide the generated predicted delay.

11. The system of claim 10, wherein the processor is further configured to: (i) generate, using the trained workflow delay prediction algorithm, one or more recommendations for preventing or alleviating the generated predicted delay; and (ii) administer the one or more recommendations to prevent or alleviate the generated predicted delay.

12. The system of claim 10, wherein the processor is further configured to store, in a database, the generated predicted delay for subsequent analysis.

13. The system of claim 10, wherein the user interface is further configured to provide a graphic representation of workflow within the ED, wherein the generated predicted delay is overlayed on the graphic representation at a location of the predicted delay within the workflow.

14. The system of claim 13, wherein the overlay of the generated predicted delay on the graphic representation comprises a color indicating the generated predicted delay.

15. The system of claim 10, wherein the user interface is further configured to provide a graphic representation of each of a plurality of patients in the ED, and an indication to which of the plurality of patients in the ED the generated predicted delay applies.
